(19) United States
(12) Patent Application Publication
(10) Pub. No.: US 2021/0103019 A1
(43) Pub. Date: Apr. 8, 2021

(54) OPEN BORE MAGNET FOR MRI GUIDED RADIOTHERAPY SYSTEM

(71) Applicant: THE UNIVERSITY OF QUEENSLAND, St Lucia, Queensland (AU)

(72) Inventors: Feng LIU, Forest Lake, Queensland (AU); Stuart CROZIER, Wilston, Queensland (AU)

(21) Appl. No.: 16/645,168

(22) PCT Filed: Sep. 5, 2018
(86) PCT No.: PCT/AU2018/050960

§ 371 Date: Mar. 6, 2020

(30) Foreign Application Priority Data
Sep. 6, 2017 (AU) 2017903603

(51) Int. Cl. G01R 33/3815; G01R 33/38; G01R 33/48; G01R 33/385

(52) U.S. Cl. CPC G01R 33/3815; G01R 33/3856; G01R 33/4808; G01R 33/3806

(57) ABSTRACT

A superconducting magnet for MRI comprising two magnet assemblies spaced along an axis and producing at least 0.7 Tesla. Each assembly including a primary coil structure (PCS) having at least first and second layers of radially-stacked primary coils and a shielding coil structure (SCS). Each layer including one or more primary coils in parallel to the axis and situated between inner and outer axial ends of the assembly that are closest to and furthest from an imaging region. The first and second layers having primary coils adjacent to the inner axial end. The PCS including a primary coil spaced from the inner axial end. The inner diameter of each primary coil of the second layer being greater than that of each primary coil in the first layer and similar to that of each coil of the SCS. The layers and shielding coil are arranged on three former portions.

### TECHNICAL FIELD

The present invention relates to actively shielded superconducting magnets for producing homogeneous magnetic fields (B₀ fields) in magnetic resonance imaging (MRI) guided radiation therapy applications.

### BACKGROUND

The aim of radiotherapy is to accurately deliver a curative dose to a tumor without damaging the surrounding normal tissue. Radiotherapy treatment is often guided by X-ray CT, which however, often gives very poor contrast between tumors and soft tissue. The advent of an integrated Magnetic Resonance imaging (MRI) system and linear accelerator (LINAC) offers improved image guidance for cancer treatment. In an MRI-LINAC hybrid system, MRI helps accurately locate tumours during a treatment session in near real time, providing greater potential of enhancing cancer treatment outcomes. Importantly, in addition to allowing real-time volumetric imaging, MRI also offers exquisite soft tissue contrast, which helps to differentiate cancerous tissues from healthy ones, thereby minimizing the radiation dose to the surrounding normal tissues and organs.

In clinical practice, MRI is a mainstream medical imaging technique used in radiology to visualize the internal structure and function of the body. MRI largely depends for its success on the generation of strong and uniform magnetic fields. A major specification of the static field in MRI is that it has to be substantially homogeneous over a predetermined region, known in the art as the “diameter spherical imaging volume” or “dsv”. The magnetic field deviations in the dsv are typically required to be less than 20 parts per million peak-to-peak (or 10 parts per million RMS).

The basic components of a typical magnetic resonance system for producing diagnostic images for human studies include a main magnet (usually a superconducting magnet which produces the substantially uniform magnetic field (the “B₀” field) in the dsv), one or more sets of shim coils, a set of gradient coils, and one or more RF coils.

Discussions of MRI, can be found in, for example, Haacke et al., Magnetic Resonance Imaging: Physical Principles and Sequence Design, John Wiley & Sons, Inc., New York, 1999. See also Crozier et. a.l., U.S. Pat. Nos. 5,818,319, 6,140,900 and 6,700,468, Dorri et al U.S. Pat. Nos. 5,396,207 and 5,416,415, Knuttel et al U.S. Pat. No. 5,646,532, and Laskaris et. al., U.S. Pat. No. 5,801,609.

A whole body MRI magnet is typically of generally annular form (i.e., in the form of a hollow cylinder or thick-walled cylindrical pipe) and arranged so that its axis of symmetry and the central opening or tunnel (referred to in the art as the “bore”) extend horizontally to receive the body of a patient. The magnets are typically around 1.6-2.0 meters in length with bore diameters in the range of 0.6-0.8 meters. Normally, the magnet is symmetric such that the midpoint of the dsv is located at the geometric center of the magnet along its longitudinal axis.

Moreover, the magnet tunnel is closed at one end, and the large distance between the portion of the patient's body which is being imaged and the open end of the magnet means that physicians cannot easily assist or personally monitor a patient during an MRI procedure.

These standard whole-body superconductive MRI magnets are usually incompatible with image-guided therapy, where a linear accelerator (“LINAC”) is used to deliver radiation therapy while the patient is simultaneously being imaged by an MRI system. To develop such an MRI-LINAC system, the MRI magnets need to be reconfigured to provide sufficient space for dual access by both the patient and a linear accelerator. However, it is further challenging to maintain high performance medical imaging in an MRI-LINAC system, because both the MRI scanner and the accelerator require electromagnetic fields to function. The resulting electromagnetic coupling between the two sub-systems restricts the orientations of the accelerator and MRI subsystems. According to the relative orientation of the medical LINAC with respect to the main magnetic field of the MRI scanner, an MRI-LINAC system can be categorized as having either an in-line configuration or a perpendicular configuration.

For example, in an MRI-LINAC system still being developed by Elekta and Philips, a high-field MRI system (1.5 Tesla) is combined with a linear accelerator, and the main magnetic field is perpendicular to the treatment beam (as described in B W Raaymakers, et. al., Integrating a 1.5 T MRI scanner with a 6 MV accelerator: proof of concept, Physics in Medicine and Biology. Phys. Med. Biol. 54 (2009) N229-N237). To achieve this perpendicular configuration, the MRI magnet was slightly modified by effectively dividing the cylindrical coils that generate the magnetic field into two cylindrical halves, and introducing a small gap of 15 cm between the resulting cylindrical (half) coils. This configuration allows the linear accelerator to be mounted on a circular gantry around the cryostat of the MRI system and directed radially inwards in the gap between the coils. However, this perpendicular configuration poses challenges in handling electromagnetic coupling between the two systems in close proximity. In particular, Lorentz force induced bending of the electron beam has to be managed, and the electron gun has to be well shielded from the MRI magnet. Otherwise, the electromagnetic interaction could degrade the functionality of radiotherapy.

In another MRI-LINAC configuration proposed by ViewRay (http://www.viewray.com), the electron beam path and the main field of the MRI system are in-line; that is, the treatment beam is oriented parallel to the MRI magnetic field direction. The ViewRay system uses a vertically-gapped (double-donut) horizontal solenoidal superconducting 0.35 Tesla whole body MRI system, and a linear accelerator is located in the fringe field. It has a large pole-pole gap (up to 60 cm), which is patient friendly; the low field strength, however, can make it difficult to provide high-resolution images for tumor tracking in real time.

It is desired to provide a magnet for an MRI system that alleviates one or more difficulties of the prior art, or to at least provide a useful alternative.

### SUMMARY

In accordance with the present invention, there is provided a superconducting magnet for an MRI system, the magnet including two magnet assemblies mutually spaced along a common axis and being configured to produce a magnetic field of at least 0.7 Tesla in an imaging region between the two magnet assemblies, each of the magnet assemblies being generally annular and disposed around a corresponding bore or opening that extends through the magnet assembly along the common axis, and including a primary coil structure having at least two layers of radially-stacked primary coils, and a shielding coil structure, each of the layers including one or more primary coils coaxial with respect to the common axis and located at one or more respective locations parallel to the common axis and between an inner axial end of the magnet assembly closest to the imaging region and an outer axial end of the magnet assembly furthest from the imaging region, wherein, in each magnet assembly:
the at least two layers include first and second layers having respective primary coils located at or adjacent to the inner axial end of the magnet assembly, and the primary coil structure includes at least one primary coil spaced from the inner axial end of the magnet assembly, and
the inner diameter of each primary coil of the second radial layer is greater than the inner diameter of each primary coil in the first layer and is similar to or less than the inner diameter of each coil of the shielding coil structure,
and wherein the first and second radial layers and the shielding coil are arranged on first, second and third former portions, respectively, surrounding the bore, and wherein the second former portion has an average inside diameter which is greater than the inside diameter of the first former portion and is similar to or less than the inside diameter of the third former portion.

In some embodiments, the primary coil at or adjacent to the inner axial end of the magnet assembly in the first radial layer has opposite current polarity to each of the primary coils in the second radial layer at or adjacent to the inner axial end of the magnet assembly.

In some embodiments, each primary coil in the second radial layer is considerably larger than any of the primary coils in the first radial layer.

In some embodiments, the shielding coil structure includes at least one shielding coil of greater diameter than the primary coils of the first layer, the shielding coil structure being located radially outwardly of the primary coils and extending approximately the axial length of first former portion of the magnet.

In some embodiments, each shielding coil has opposite current polarity to the primary coils of the second layer and a majority of the primary coils of the first layer.

In some embodiments, the magnet includes a LINAC system to form a hybrid MRI-LINAC apparatus wherein a patient in the imaging region can be arranged such that a longitudinal axis of the patient is either co-linear with or orthogonal to the common axis of the magnet and the LINAC system produces a beam that is orthogonal to the longitudinal axis of the patient.

The patient may be located at an isocenter of the hybrid MRI-LINAC apparatus.

In some embodiments, the coils form a low field strength region of ≤0.2 Tesla at locations on the axis of the magnet proximal to the MRI-LINAC apparatus to allow an electron gun of the LINAC to operate in the presence of an aligned MRI magnet fringe field.

In some embodiments, a dimension of the central gap in the axial direction is at least 30 cm to allow for dual simultaneous access by a patient and the LINAC system.

In some embodiments, the inner diameter of the primary coils of the first radial layer is between 20 cm and 100 cm.

In some embodiments, each magnet assembly has a cold bore axial length less than 100 cm.

In some embodiments, a dimension of the imaging region in the axial direction is at least 20 cm.

In some embodiments, the magnet further comprises a split gradient coil structure having gradient coils mounted along respective bores of the respective magnet assemblies.

In some embodiments, the magnet assemblies are cooled by a common cryogenic system. In some embodiments, the common cryogenic system is longitudinally disposed between the magnet assemblies where no windings or electrical connections are present.

In accordance with the present invention, there is provided a magnetic resonance imaging system having any of the above magnets.

Embodiments of the present invention provide a high-field superconducting magnet suitable for use in a MRI system for imaging of a tumour in the human body, providing real-time guidance for radiation therapy.

As described herein, the magnet is actively shielded and wound in a split-pair configuration of mutually spaced magnet assemblies. The two magnet assemblies share the same magnetic axis and are capable of producing a magnetic field of at least 0.7 Tesla in an imaging region located in the central gap between the two magnet assemblies. The magnet allows a LINAC beam operating in an in-line orientation with respect to the MRI magnetic field, the magnet configuration, however, can also be used for a radial LINAC configuration, where the beam is perpendicular to the MRI magnetic field direction.

An advantage of having a ‘dual-bore’ magnet configuration is that the dsv is located in the centre of the gap, allowing for the patent's access and movement, for example, rotation on the patient bed around the main magnet axis during radiation treatment. In addition, the split bore also minimizes the sense of claustrophobia experienced by patients. It is noted that in the in-line setting, the orientation of the patient with respect to the MRI scanner is orthogonal to the magnetic field and to the conventional position in a clinical MRI scan.

Each magnet assembly comprises a structure of three radially layered coils, including a primary coil structure formed by the first and second radial layers, and a shielding coil structure formed by the third radial layer.

The first layer of the primary coil structure includes at least first, second and third sets of coils coaxially aligned and positioned along the longitudinal axis of the magnet assemblies, each set of coils having a smaller inner diameter to the other sets.

A primary coil in the first layer is located adjacent to a first axial end of the magnet closest to the imaging region, a primary coil in the second or third set is located adjacent to a second axial end of the magnet being opposite to the first axial end and furthest from the imaging region, and for the case with three primary coil sets, the second set is located between the first and third sets of primary coils. In the first layer, the inner diameter of each coil set is less than the inner diameter of each coil of the second and third layers.

In the second layer, the inner diameter of each coil set is larger than the inner diameter of each coil of the first layer, but similar to or less than the inner diameter of the or each coil of the third layer. Preferably, the coil size (cross section) of the second layer is substantially larger than that of the coils in the first layer.

Typically, the third layer contains shielding coils which have opposite current polarity to the majority of the primary coils. Preferably, the outer diameter of the or each coil of the third layer is similar or larger than those of the second layer, and considerably greater than the ones located at the first layer.

In the described embodiments, each magnet assembly is provided with a three-layered former structure, which is preferably cylindrically shaped, having at least three former portions or segments, for the respective coil sets. Each of the first, second and third sets of coils are arranged on first, second and third former portions or segments, respectively, surrounding the bore. Preferably, the outer diameter of the first former segment is smaller than the outer diameter of the second former segment which, in turn, is smaller than the outer diameter of the third former segment.

In some embodiments, a split gradient coil is provided for the magnet, with a first part of the gradient coil mounted along the bore of the first magnet assembly, and a second part of the gradient coil mounted along the bore of the second magnet assembly.

In an embodiment, the central gap of the magnet along the longitudinal axis (i.e., the spacing between the two magnet assemblies) is larger than 30 cm and less than 80 cm.

The magnet preferably has a cold bore axial length less than 100 cm for each split bore, and the dimension of the imaging region along the axial direction is preferably at least 20 cm.

A shielding coil structure is preferably provided radially around the primary coil structure, extending approximately the axial length of the bore of the magnet. The shielding coil structure forms layer 3 of the magnet and may have its own former, and has at least one shielding coil of greater diameter than the primary coils.

Preferably, force balancing is used in the design of the magnet to minimize or at least reduce the net forces on the coils. In implementing the step of force balancing, Maxwell forces are included in an error function to be minimized.

The two halves of the magnet may be cooled using one or two cryogenic systems across the central section where no windings or electrical connections are present.

In some embodiments, the magnet stray fields include a low field region (≤0.06 Tesla) close the magnet end that is opposite to the one close to dsv. The size of the low-field region (in both radial and axial directions) is large enough to accommodate the LINAC system.

In some embodiments, the magnet produces a magnetic field of at least 0.7 Tesla in the imaging region between the two magnet assemblies, and each magnet assembly includes four primary coils, two of the primary coils being disposed at an inner axial end of the magnet assembly in respective first and second layers, and the other two primary coils being spaced from the inner axial end of the magnet assembly by respective distances.

In some embodiments, the magnet produces a magnetic field of at least 0.7 Tesla in the imaging region between the two magnet assemblies, and each magnet assembly includes five primary coils, three of the primary coils being disposed at an inner axial end of the magnet assembly in respective first, second and third layers, and the other two primary coils being spaced from the inner axial end of the magnet assembly by respective distances.

In some embodiments, the magnet produces a magnetic field of at least 1.0 Tesla in the imaging region between the two magnet assemblies, and each magnet assembly includes three primary coils, two of the primary coils being disposed at an inner axial end of the magnet assembly in respective first and second layers, and the other primary coil being spaced from the inner axial end of the magnet assembly and closer to an outer axial end of the magnet assembly.

In some embodiments, the magnet produces a magnetic field of at least 1.5 Tesla in the imaging region between the two magnet assemblies, and each magnet assembly includes three primary coils, two of the primary coils being disposed at an inner axial end of the magnet assembly in respective first and second layers, and the other primary coil being spaced from the inner axial end of the magnet assembly and closer to an outer axial end of the magnet assembly.

In another form, the invention provides a magnetic resonance imaging system having a magnet as described above.

The above summary of the invention and certain embodiments are only for the convenience of the reader, and are not intended to and should not be interpreted as limiting the scope of the invention. More generally, it is to be understood that both the foregoing general description and the following detailed description are merely exemplary of the invention, and are intended to provide an overview or framework for understanding the nature and character of the invention as it is claimed.

Additional features and advantages of the invention are set forth in the detailed description which follows. Both these additional features of the invention and those discussed above can be used separately or in any and all combinations.

### BRIEF DESCRIPTION OF THE DRAWINGS

Some embodiments of the present invention are hereinafter described, by way of example only, with reference to the accompanying drawings, in which like reference numbers refer to like parts, and wherein:

FIG. 1B is a schematic illustration of an MRI-LINAC system with a split-bore superconducting magnet in accordance with the described embodiments of the present invention.

FIG. 1A is a cross-section side view of a superconducting magnet for an MRI system in accordance with a first embodiment of the present invention.

FIGS. 2A, 2B and 2C are schematic cross-sectional side views showing the coil configurations and dsv size of three different 0.7 T superconducting magnets in accordance with second, third and fourth embodiments of the present invention, respectively.

FIG. 3 is a cross-section side view showing the coil configuration and dsv size of a 1.0 T superconducting magnet in accordance with a fifth embodiment of the present invention.

FIG. 4 is a cross-section side view showing the coil configuration and dsv size of a 1.5 T magnet in accordance with a sixth embodiment of the present invention.

FIGS. 5A, B, and C are contour plots of stray magnetic fields outside the magnets of FIGS. 2A, 2B, and 2C, respectively, specifically the 5 Gauss (5×10⁴ Tesla), 60 mT and 100 mT contours as a function of longitudinal and radial distances.

FIG. 6 is a contour plot of the stray field outside the 1.0 T magnet of FIG. 3 as a function of longitudinal and radial distances, specifically the 5 gauss (5×10⁻⁴ Tesla), 60 mT and 100 mT contours.

FIG. 7 is a contour plot of the stray field outside the 1.5 T magnet of FIG. 4 as a function of longitudinal and radial distances, specifically the 5 gauss (5×10⁻⁴ Tesla), 60 mT and 100 mT contours.

FIGS. 8A, 8B, and 8C show contours of the calculated magnitudes of the total magnetic field within the coils of the 0.7 T magnets of FIGS. 2A, 2B, and 2C, respectively.

FIG. 9 shows contours of the calculated magnitudes of the total magnetic field within the coils of the 1.0 T magnet of FIG. 3.

FIG. 10 shows contours of the calculated magnitudes of the total magnetic field within the coils of the 1.5 T magnet of FIG. 4.

FIGS. 11A, 11B, and 11C show contours of calculated magnitudes of the total electromagnetic forces within the coils of the 0.7 T magnet of FIGS. 2A, 2B, and 2C, respectively.

FIG. 12 shows contours of calculated magnitudes of the total electromagnetic forces within the coils of the 1.0 T magnet of FIG. 3.

FIG. 13 shows contours of calculated magnitudes of the total electromagnetic forces within the coils of the 1.5 T magnet of FIG. 4.

### DETAILED DESCRIPTION

Embodiments of the present invention provide a superconducting magnet for an MRI system. The magnet includes two generally annular magnet assemblies mutually spaced along a common axis to define a gap and imaging region therebetween. The annular shape of each magnet assembly defines a corresponding central opening or “bore” that extends through the magnet assembly, and because the magnet is effectively divided or split into two mutually spaced assemblies, so too the magnet bore can be considered to be divided or split and is thus described herein as a ‘split bore’. Each magnet assembly has a primary coil structure comprising radially-stacked layers of primary coils arranged around the bore. The primary coil structure is surrounded by a shielding coil structure or layer made up of an arrangement of one or more shielding coils. The shielding coils are used to reduce the stray magnetic field to a desired level (typically, ≤5 Gauss) within a specified space/region (in the described embodiments being a region extending to a distance of about 5 m from the magnet center).

The primary coil structure includes at least two layers of primary coils with significantly different inner diameters, as illustrated schematically in the drawings. Each of these layers includes a corresponding primary coil located at or adjacent to a first or inner axial end of the magnet assembly closest to the imaging region and the gap between the two magnet assemblies. Each magnet assembly also includes at least one primary coil spaced from the inner axial end of the magnet assembly, and in some embodiments is located at or adjacent to a second or ‘outer’ axial end of the magnet assembly opposite to the first axial end and furthest from the imaging region. The two radial primary coil layers and the shielding coil are arranged on respective (first, second, and third) former portions surrounding the bore, wherein the second former portion has a minimum inside diameter which is greater than the minimum inside diameter of the first former portion but similar to or less than the minimum inside diameter of the third former portion.

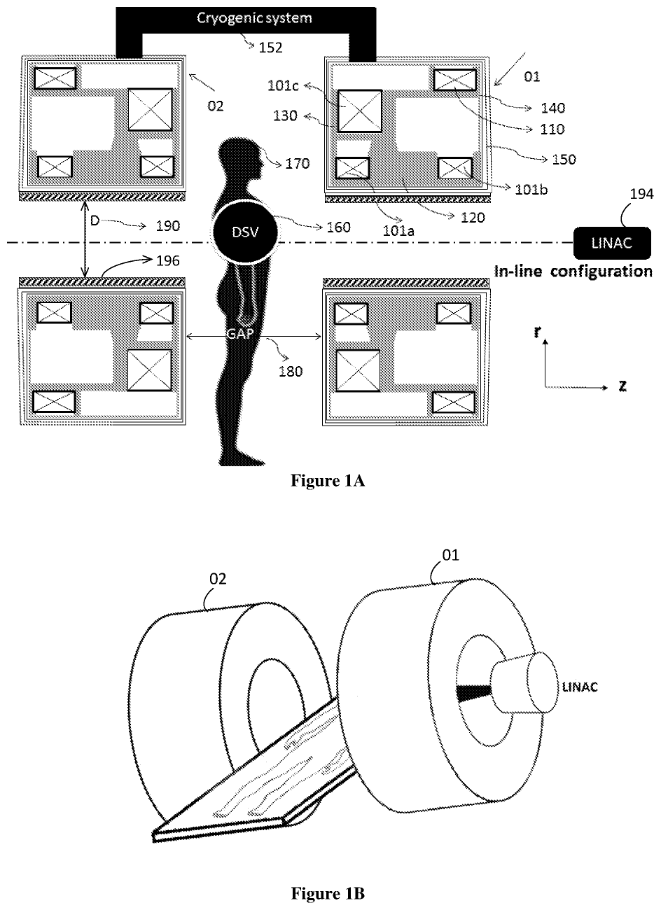

FIGS. 1A and B are schematic representations of an embodiment of the superconducting magnet in the context of an MRI-LINAC apparatus or system. In this dedicated open bore MRI system, a patient is positioned in the magnet center, in a perpendicular position with respect to the main magnetic field (along the longitudinal axis of the magnet), while the treatment beam is in-line or collinear with the main magnetic field of the MRI magnet. During treatment, the patient between the two magnet assemblies can be rotated about the longitudinal axis of the magnet. Alternatively, as the magnet has a large bore in the longitudinal direction, the patient can be positioned in parallel with the main magnetic field.

In the primary coil structure of the magnet, the two primary coil layers are wound on respective former segments having different inner diameters or bores. These two former segments are interconnected in series to construct a magnet structure aligned coaxially with a longitudinal axis of the magnet. Materials of the two former segments can be either metal such as, but not limited to, non-magnetic stainless steel, or non-metal such as, glass fibre reinforced polymer (GFRP).

In the described embodiments, to generate linear spatial variations of magnetic fields in the imaging region (for MRI signal encoding) and also to reduce the stray fields in the magnet bore, split gradient coils are actively-shielded and mounted in the magnet cryostat with a central, axial gap to accommodate a linear accelerator (LINAC) and a patient.

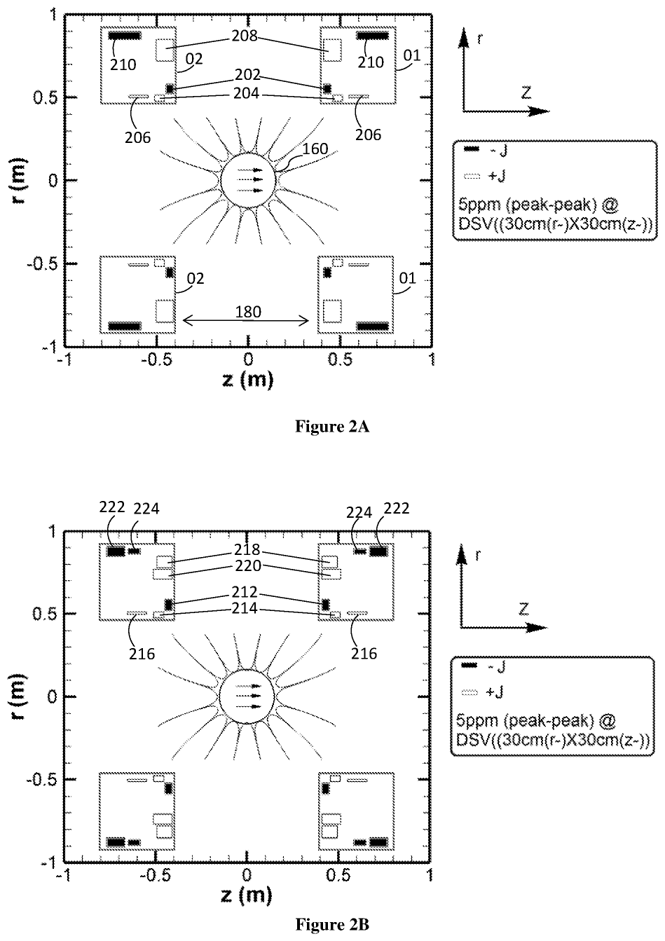

FIG. 1 shows a first embodiment of a superconducting magnet for an MRI system. In each of two mutually spaced magnet assemblies 01, 02, a first primary coil layer of two (but not limited to two) superconductive primary coils 101a and 101b having the same or similar inner diameters are wound around a cylindrically-shaped first former segment 120. In a second primary coil layer, a single (but not limited to one) further superconductive primary coil 101c is wound around a cylindrically-shaped second former segment 130. Each second layer primary coil has a larger inner diameter than the inner diameters of the first layer primary coils 101a and 101b. Each of the first and second primary coil layers includes a corresponding primary coil located at or adjacent to one (axial) end of each magnet assembly that, when installed, is the end of that assembly that is closest to the imaging region or dsv 160 and the center of the magnet. In various embodiments, the primary coil structure of each magnet assembly 01, 02 always includes one or more further primary coils spaced from the first axial end, namely: (i) at or adjacent to a second axial end of the magnet assembly opposite to the first axial end (i.e., at the outer ends of the magnet assemblies 01, 02 furthest from the imaging region or dsv 160), in the embodiment of FIG. 1 being the primary coil 101b of the first layer, and/or (ii) at one or more axial locations between the first and second axial ends of each magnet assemblies 01, 02, as shown in FIGS. 2A, 2B and 2C, for example.

One (but not limited to one) superconductive shielding coils 110, having opposite current polarity to the majority of the primary coils 101a, 101b, 101c, are wound around a shield former 140, so as to reduce the stray magnetic field to a desired level (typically, ≤5 Gauss).

Each magnet assembly 01, 02 includes a corresponding vacuum chamber 150 containing all of the corresponding primary 101a, 101b, 101c and shielding 110 coils and the corresponding formers 120, 130, 140. Both vacuum chambers 150 are interconnected and cooled by a common cryogenic system 152 such that the vacuum chambers 150 and the cryogenic system collectively constitute a common vacuum chamber.

Although the magnet may be used for non MRI-LINAC specific applications, such as interventional imaging, for example, it has been designed for MRI-LINAC applications and generates a magnetic field strength of at least 0.7 Tesla within a diameter of spherical volume (‘dsv’) 160 which is located in the central gap of the magnet 01. The first and second magnet assemblies 01 and 02 and the gap 180 therebetween are preferably dimensioned so that a typical patient 170 fits radially between the magnet assemblies 01 and 02 and/or axially inside the bore or tunnel of the magnet assemblies, characterized by the bore diameter D 190.

Compared to known magnets for MRI-LINAC applications, the described embodiments of the present invention:
(1) provide a split superconducting magnet with an open or split bore configuration that allows dual and simultaneous access for a patient 170 and a LINAC system 194;
(2) use a divided or ‘split’ gradient coil 196 to generate linear spatial variations of magnetic fields for MRI signal encoding. In conventional MRI systems, gradient coils in the shape of a hollow cylinder are inserted into the closed, cylindrical tunnel. In the MRI-LINAC system described herein, the gradient coils 196 are configured with similar shapes as their corresponding superconducting magnet assembly 01, 02. That is, the split, actively-shielded, gradient coils 196 are mounted in the inner bore adjacent the magnet wall with a central, axial gap to accommodate the accelerator 194 and the patient 170; and
(3) have an outer vacuum chamber comprising two portions 150.

Embodiments of the invention provide magnets that achieve at least some and, most preferably, all of the following performance criteria:
(1) an outer shielding coil 110 with a radius that is less than or equal to 110 cm, and preferably less than or equal to 100 cm;
(2) the cold bore length of each magnet assembly 01, 02 is less than or equal to 100 cm;
(3) a dsv 160 with dimensions of at least 20 cm(diameter)×20 cm(z) with a homogeneity of +/−10 ppm after shimming;
(4) relatively low peak magnetic fields within the coils 101a, 101b, 101c, 110 to allow for the use of less expensive superconducting wire, specifically a calculated peak magnetic field within the current carrying coils whose magnitude is less than or approximately equal to 7.5 Tesla);
(6) low stray fields, namely a calculated stray magnetic field external to the magnet that is less than 5×10⁻⁴ Tesla at all locations greater than about 5.5 meters from the geometrical centre of the dsv 160); and
(7) low stray fields in the region located proximal to the in-line MRI-LINAC apparatus to accommodate the LINAC system 194, namely a calculated stray magnetic field external to the magnet that is less than 6×10⁻² Tesla on the longitudinal axis at a distance of 1.1 meters from the dsv 160 geometrical centre).

Examples of magnets of the invention, and current distribution functions of the magnets, will now be described, without limiting the scope of the invention.

The coil positions described herein were (and other configurations can be) determined by an optimization process using a constrained numerical optimization technique based on a nonlinear least-square algorithm (see, for example, Matlab optimization toolbox, http://www.mathworks.com). The optimization process used the geometry and positions of the field generating elements as parameters and minimized a cost function that includes deviation of the magnetic fields inside the dsv, stray external fields around the magnet, peak fields and electromagnetic forces inside the coil blocks (for threshold values, see FIGS. 2 to 13) to calculate the final coil geometry for each magnet.

### EXAMPLE 1

### 0.7 Tesla Magnet

FIGS. 2A, 2B and 2C illustrates the spatial arrangements of primary and shielding magnet coils and the dsv in three respective embodiments of 0.7 T superconducting magnets. In the embodiment shown in FIG. 2A, the magnet employs four primary coils 202 to 208 (three primary coils 202 to 206 on a first former segment (not shown), and one other primary coil 208 on a second former segment (not shown)), and one shielding coil 210.

In the embodiment shown in FIG. 2B, each of the magnet assemblies 01, 02 employs five primary coils 212 to 220 (three coils 212 to 216 at the first former segment, two other coils 218, 220 at the second former segment) and two shield coils 222, 224. In the embodiment shown in FIG. 2C, the magnet employs four primary coils 226 to 232 (two coils 226, 228 on a first former segment, two other coils 230, 232 on a second former segment), and one shielding coil 234. The solid and absence of shading represent respective opposite polarities of electrical current (+J/−J: positive/negative currents in terms of contribution to the magnet fields in the dsv 160) flowing through the various coils 202 to 234.

In broad overview, all of the magnets of FIG. 2 have a cold bore length of approximately 0.5 meters, and cold bore inner and outer radii of approximately 0.43 and 0.95 meters, respectively. The magnets have a dsv 160 which is approximately spherical with a diameter of approximately 30 centimetres (magnetic field uniformity: 5 ppm peak-peak). The axial dimension of the central gap 180 between the two spaced magnet assemblies 01, 02 of each magnet is about 70 cm.

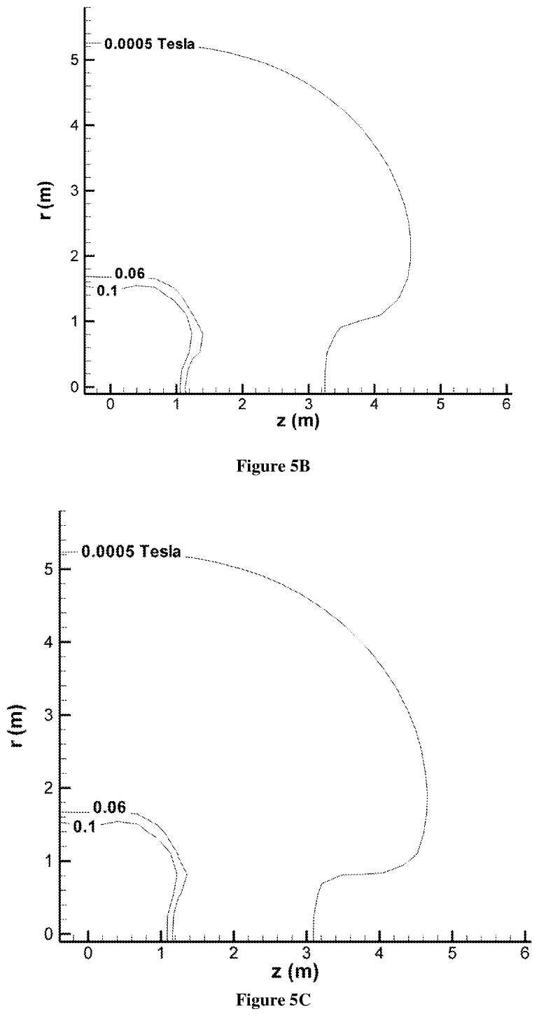

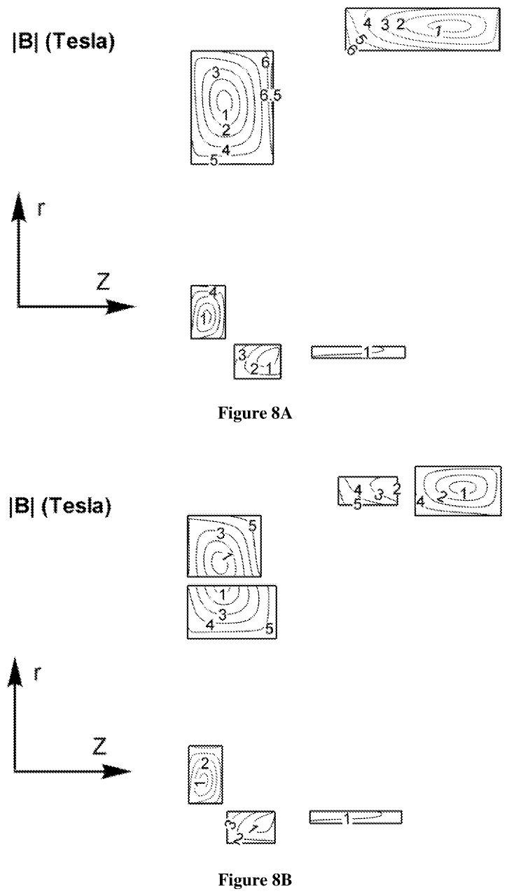

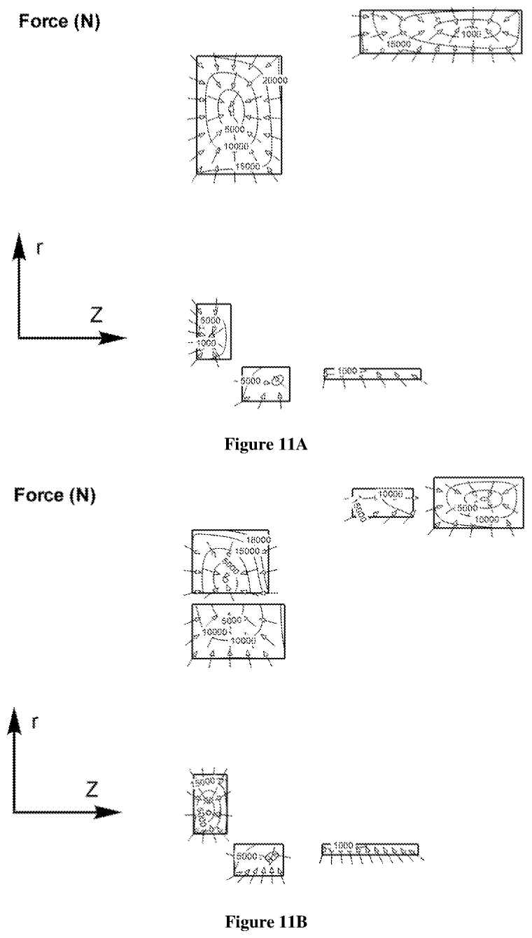

FIG. 5 shows contour plots of the corresponding calculated stray external fields generated by the respective magnets of FIG. 2, FIG. 8 shows the corresponding calculated magnitudes (in Tesla) of the total magnetic fields generated within the various coils, and FIG. 11 shows the calculated magnitudes of the total electromagnetic forces (in Newtons) generated within the various coils.

As shown in FIG. 5, all three magnets of FIG. 2 have a 5 Gauss contour line which is located approximately 4.5 m axially and 5.2 m radially from the center of the dsv 160, and produce a low stray magnetic field (of less than 6×10⁻² Tesla) at distances on the longitudinal axis greater than 1.1 meters from the dsv 160 geometrical centre.

As shown in FIG. 8, the peak calculated magnetic fields of all of the three embodiments of FIG. 2 are about 6.5 Tesla, 5 Tesla and 6 Tesla, respectively, which allows these magnets to be constructed using standard and readily available superconducting wire.

### EXAMPLE 2

### 1.0 T Magnet

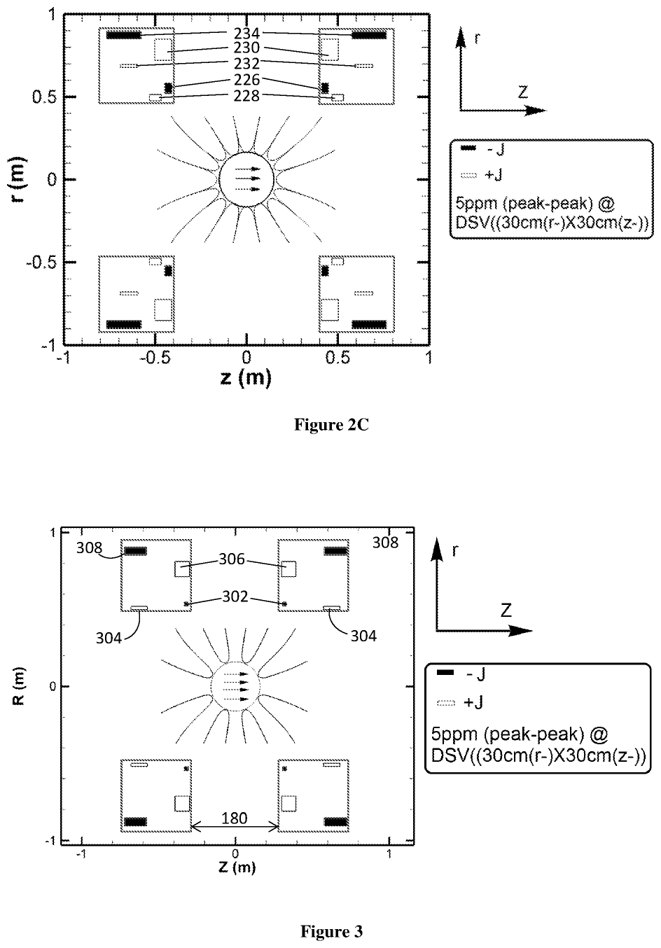

FIG. 3 illustrates the spatial arrangements of primary and shielding magnet coils and the dsv 160 in a 1.0 Tesla superconducting magnet according to another embodiment of the present invention. The magnet employs three primary coils 302, 304, 306 (two coils 302, 304 on a first former segment (not shown), one other coil 306 on a second former segment (not shown, but whose diameter is larger than that of the first former), and one shielding coil 308. In broad overview, each magnet assembly 01, 02 of the magnet has a cold bore length of approximately 0.57 meters, and cold bore inner and outer radii of approximately 0.45 and 0.95 meters, respectively. The magnet has a dsv 160 which is approximately spherical with a diameter of approximately 30 centimetres. The axial dimension of the central gap 180 is about 50 cm.

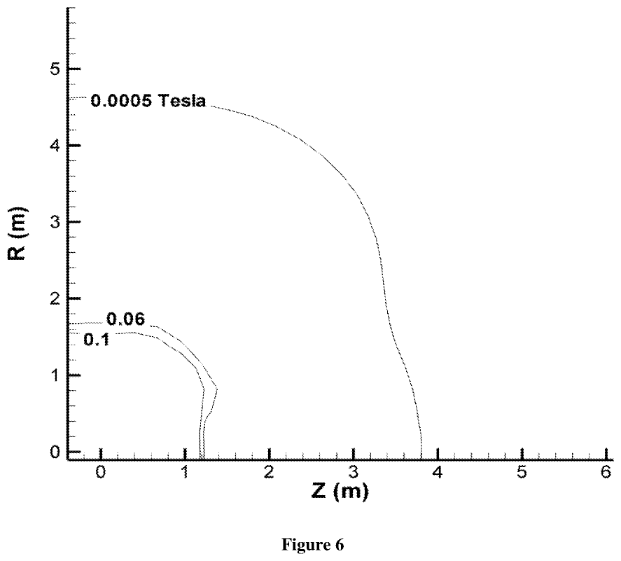

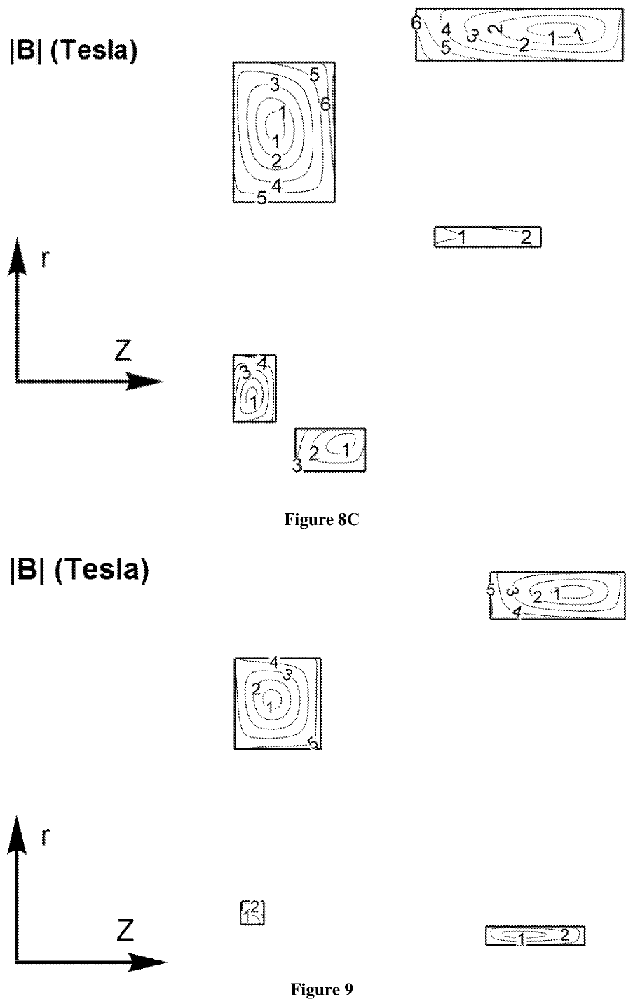

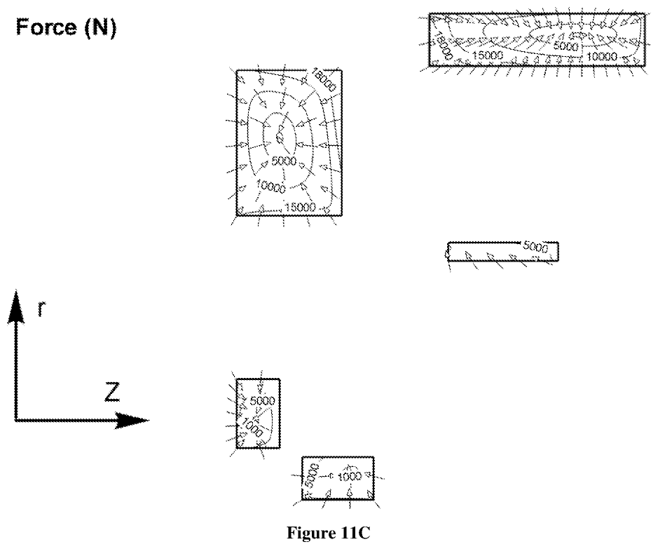

FIG. 6 is a contour plot of the corresponding calculated stray external fields generated by the magnet of FIG. 3. FIG. 9 shows the calculated magnitudes of the total magnetic field generated by the magnet within the magnet's various coils. FIG. 12 shows the calculated magnitudes of the total electromagnetic forces generated by the magnet within the magnet's various coils.

As shown in FIG. 6, the magnet of FIG. 3 produces a 5 Gauss field at a distance of approximately 3.8 m axially and 4.6 m radially from the center of the dsv 160, and a low stray magnetic field of less than 6×10⁻² Tesla on the longitudinal axis at distances greater than 1.1 meters from the dsv 160 geometrical centre. As shown in FIG. 9, the peak calculated magnetic field is about 5 Tesla, which allows the magnet to be constructed using standard and readily available superconducting wire.

### EXAMPLE 3

### 1.5 T Magnet

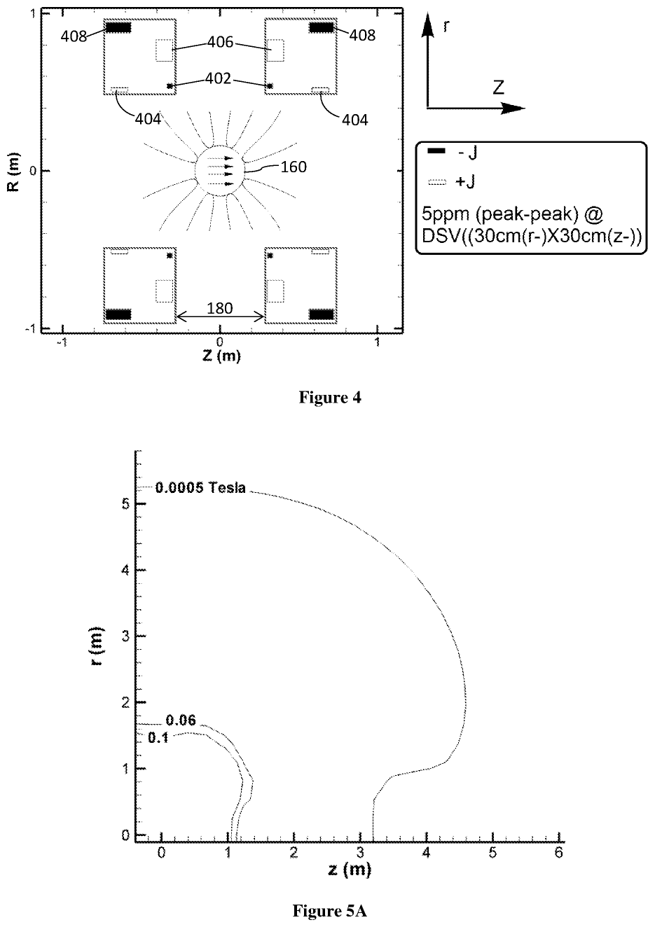

FIG. 4 shows the spatial arrangements of primary and shielding magnet coils and the dsv 160 in a 1.5 T superconducting magnet according to a further embodiment of the present invention.

Each magnet assembly 01, 02 employs three primary coils 402, 404, 406 (two primary coils 402, 404 on a first former segment (not shown), and one other primary coil 406 on a second former segment (not shown), and one shielding coil 408. In broad overview, each magnet assembly 01, 02 of the magnet has a cold bore length of approximately 0.62 meters, and a cold bore inner and outer radii of approximately 0.45 and 1.0 meters, respectively. The magnet has a dsv 160 which is approximately spherical with a diameter of approximately 30 centimetres. The axial dimension of the central gap 180 between the magnet assemblies is about 40 cm.

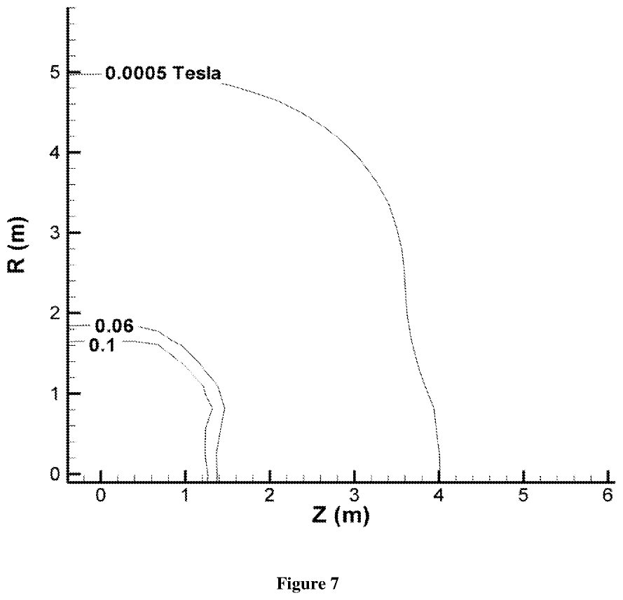

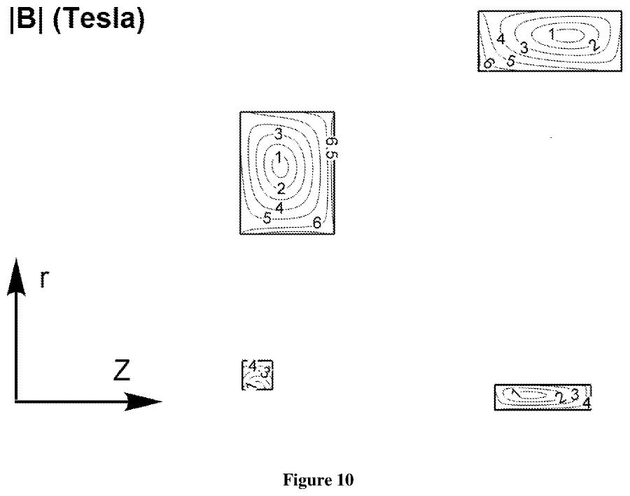

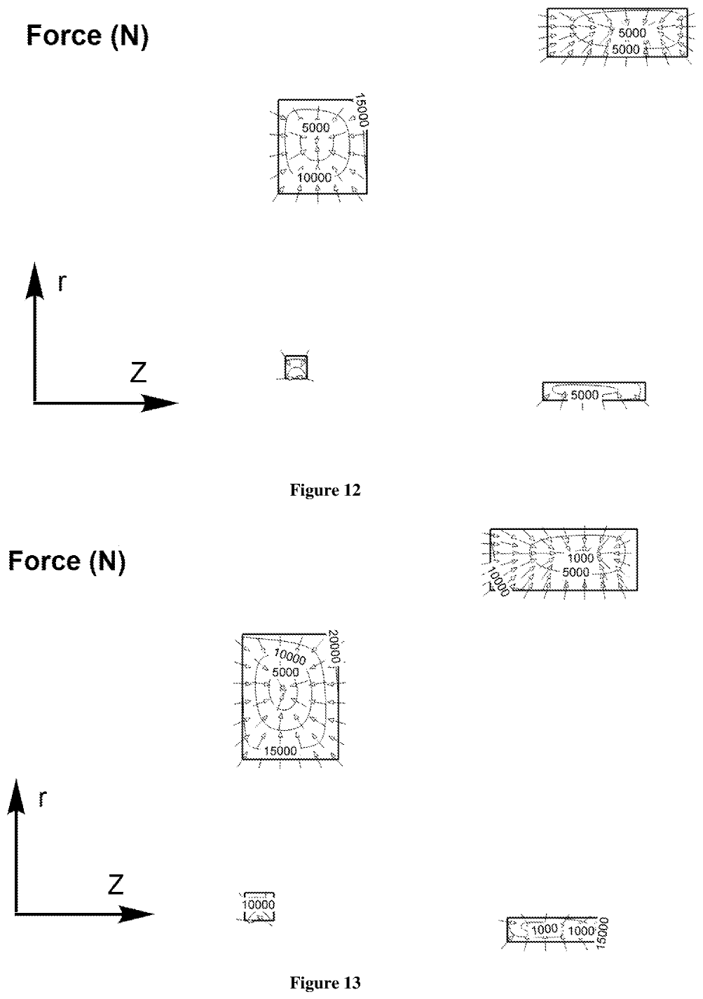

FIG. 7 shows the corresponding calculated stray external fields generated by the magnet of FIG. 4. FIG. 7 shows the calculated magnitudes of the total magnetic field generated by the magnet within the magnet's various coils. FIG. 10 shows the calculated magnitudes of the total electromagnetic forces generated by the magnet within the magnet's various coils. FIG. 13 shows the calculated magnitudes of the total electromagnetic forces generated by the magnet within the magnet's various coils.

As shown in FIG. 7, the magnet produces a field of 5 Gauss at a distance of approximately 4 m axially and 5 m radially from the center of the dsv 160, and a low stray magnetic field of less than 6×10⁻² Tesla on the longitudinal axis at distances greater than 1.1 meters from the dsv 160 geometrical centre. As shown in FIG. 10, the peak calculated magnetic field is about 6.5 Tesla, which allows the magnet to be constructed using standard and readily available superconducting wire.

The foregoing embodiments and examples are intended to be illustrative of the invention, without limiting the scope thereof. The invention is capable of being practised with various modifications and additions as will readily occur to those skilled in the art.

Where suitable or appropriate, one or more features of one embodiment may be used in combination with one or more features of another embodiment.

1. A superconducting magnet for an MRI system, the magnet including two magnet assemblies mutually spaced along a common axis and being configured to produce a magnetic field of at least 0.7 Tesla in an imaging region between the two magnet assemblies, each of the magnet assemblies being generally annular and disposed around a corresponding bore or opening that extends through the magnet assembly along the common axis, and including a primary coil structure having at least two layers of radially-stacked primary coils, and a shielding coil structure, each of the layers including one or more primary coils coaxial with respect to the common axis and located at one or more respective locations parallel to the common axis and between an inner axial end of the magnet assembly closest to the imaging region and an outer axial end of the magnet assembly furthest from the imaging region, wherein, in each magnet assembly:
the at least two layers include first and second layers having respective primary coils located at or adjacent to the inner axial end of the magnet assembly, and the primary coil structure includes at least one primary coil spaced from the inner axial end of the magnet assembly, and
the inner diameter of each primary coil of the second radial layer is greater than the inner diameter of each primary coil in the first layer and is similar to or less than the inner diameter of each coil of the shielding coil structure,
and wherein the first and second radial layers and the shielding coil are arranged on first, second and third former portions, respectively, surrounding the bore, and wherein the second former portion has an average inside diameter which is greater than the inside diameter of the first former portion and is similar to or less than the inside diameter of the third former portion.

2. The magnet of claim 1, wherein the primary coil at or adjacent to the inner axial end of the magnet assembly in the first radial layer has opposite current polarity to each of the primary coils in the second radial layer at or adjacent to the inner axial end of the magnet assembly.

3. The magnet of claim 1, wherein each primary coil in the second radial layer is considerably larger than any of the primary coils in the first radial layer.

4. The magnet of claim 1, wherein the shielding coil structure includes at least one shielding coil of greater diameter than the primary coils of the first layer, the shielding coil structure being located radially outwardly of the primary coils and extending approximately the axial length of first former portion of the magnet.

5. The magnet of claim 4, wherein each shielding coil has opposite current polarity to the primary coils of the second layer and a majority of the primary coils of the first layer.

6. The magnet of claim 1, including a LINAC system to form a hybrid MRI-LINAC apparatus wherein a patient in the imaging region can be arranged such that a longitudinal axis of the patient is either co-linear with or orthogonal to the common axis of the magnet and the LINAC system produces a beam that is orthogonal to the longitudinal axis of the patient.

7. The magnet and MRI system of claim 6, wherein said patient is located at an isocenter of the hybrid MRI-LINAC apparatus.

8. The magnet of claim 4, wherein the coils form a low field strength region of <0.2 Tesla at locations on the axis of the magnet proximal to the MRI-LINAC apparatus to allow an electron gun of the LINAC to operate in the presence of an aligned MRI magnet fringe field.

9. The magnet of claim 6, wherein a dimension of the central gap in the axial direction is at least 30 cm to allow for dual simultaneous access by a patient and the LINAC system.

10. The magnet of claim 1, wherein the inner diameter of the primary coils of the first radial layer is between 20 cm and 100 cm.

11. The magnet of claim 1, wherein each magnet assembly has a cold bore axial length less than 100 cm.

12. The magnet of claim 1, wherein a dimension of the imaging region in the axial direction is at least 20 cm.

13. The magnet of claim 1, further comprising a split gradient coil structure having gradient coils mounted along respective bores of the respective magnet assemblies.

14. The magnet of claim 1, wherein the magnet assemblies are cooled by a common cryogenic system.

15. The magnet of claim 14, wherein the common cryogenic system is longitudinally disposed between the magnet assemblies where no windings or electrical connections are present.

16. A magnetic resonance imaging system having a magnet as claimed in claim 1.
